(19) United States
(12) Patent Application Publication
(10) Pub. No.: US 2016/0022346 A1
(43) Pub. Date: Jan. 28, 2016

(54) MEDICAL DEVICE AND METHOD OF USE

(71) Applicant: Cirrus Technologies KFT, Budapest (HU)

(72) Inventor: John H. SHADDUCK, Menlo Park, CA (US)

(73) Assignee: Cirrus Technologies KFT, Budapest (HU)

(21) Appl. No.: 14/789,830

(22) Filed: Jul. 1, 2015

Related U.S. Application Data
(60) Provisional application No. 62/019,813, filed on Jul. 1, 2014.

(51) Int. Cl. A61B 18/02

(52) U.S. Cl. CPC A61B 18/0218; A61B 2018/00005

(57) ABSTRACT

Systems and methods for treating tissue with cryogenic media and/or high pressure jet media for cutting either sequentially or contemporaneously and removing tissue from a site in a patient body.

### CROSS-REFERENCE TO RELATED APPLICATIONS

This application claims benefit of priority to U.S. Provisional Patent Application No. 62/019,813, filed on Jul. 1, 2014, the content of which is incorporated herein by reference in its entirety.

### FIELD OF THE INVENTION

The present invention relates to a system for treating tissue contemporaneously with cryogenic media and/or high pressure jet media for cutting and removing tissue from a site in a patient body that includes a moveable discharge nozzle for discharging the jetted fluid media.

### BRIEF DESCRIPTION OF THE DRAWINGS

The disclosed variations will next be described in greater detail by reference to exemplary embodiments that are illustrated in the drawings.

FIG. 1 is a side view of one variation of a tissue resecting probe for use as described herein.

FIG. 2 is a cut-away representation of a distal end of the probe of FIG. 1.

FIG. 3 is a sectional view of the distal end of the probe as in FIG. 2 taken along, line X-X of FIG. 2.

FIG. 4A shows an endoscope introduced trans-urethrally into a patient prostate, and a resecting probe being introduced through a working channel of the endoscope with an optional guidewire inserted into a prostate lobe through the resecting probe.

FIG. 4B shows a resecting probe being advanced over a guidewire into the prostate lobe.

FIG. 4C shows a representation of the resecting probe activated to cause a high pressure jet of fluid media to resect tissue in the targeted site.

### DETAILED DESCRIPTION

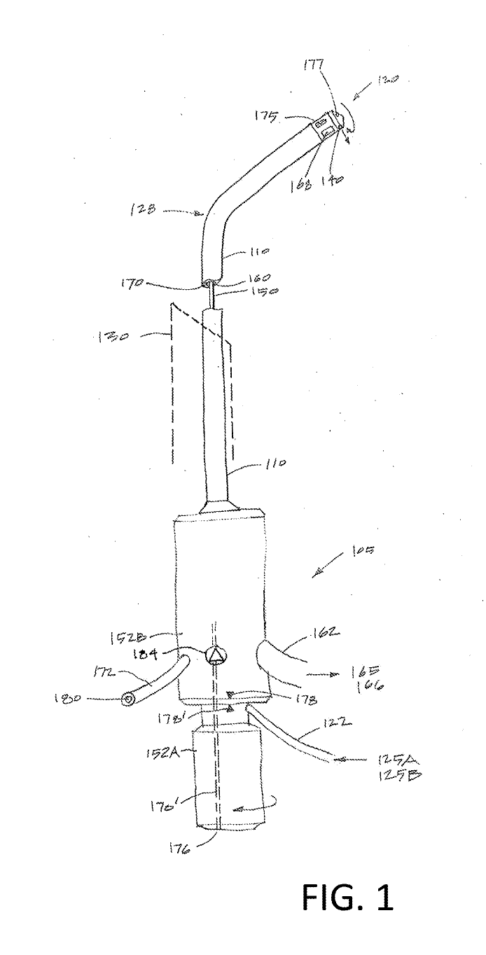

FIG. 1 shows a tissue-resecting jetting surgical instrument 100 for use in a transurethral resection of the prostate (TURP) procedure. The instrument 100 of FIG. 1 has a handle portion 105 that is coupled to an elongated shaft or probe sleeve 110 that extends to the distal working end 120. A high pressure source 125A of at least one flow media and controller 125B are provided to supply a resecting fluid to the working end 120 by supply line 122.

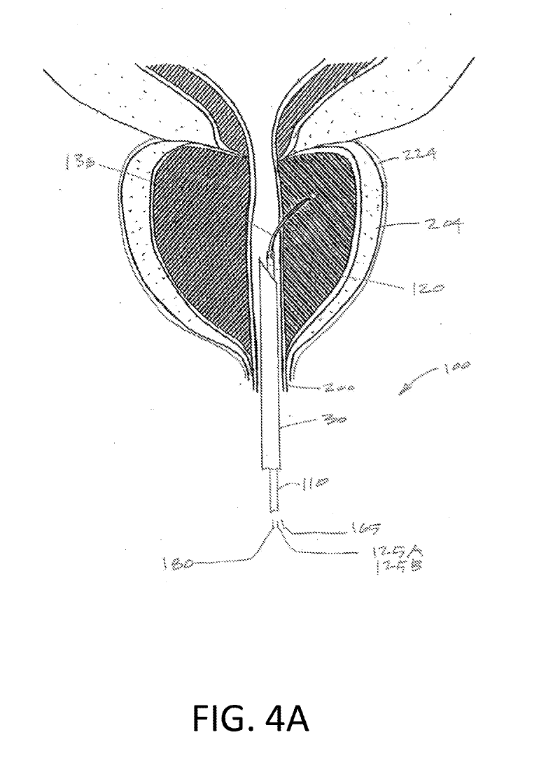

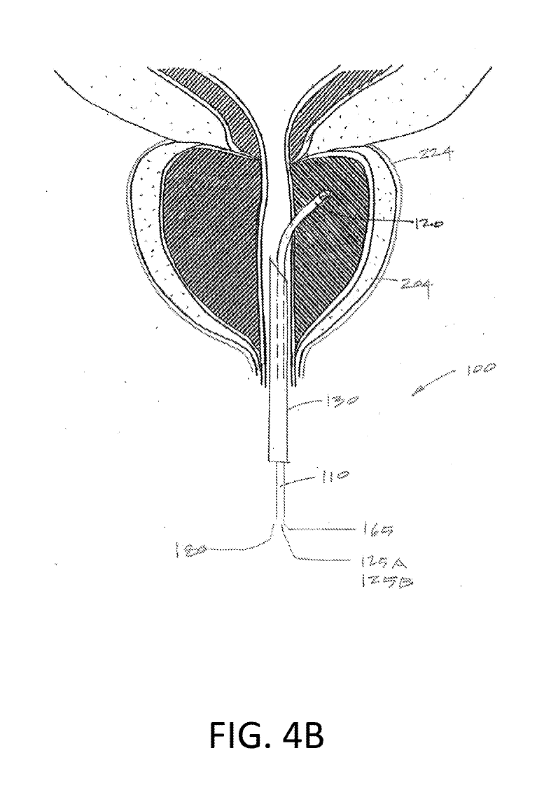

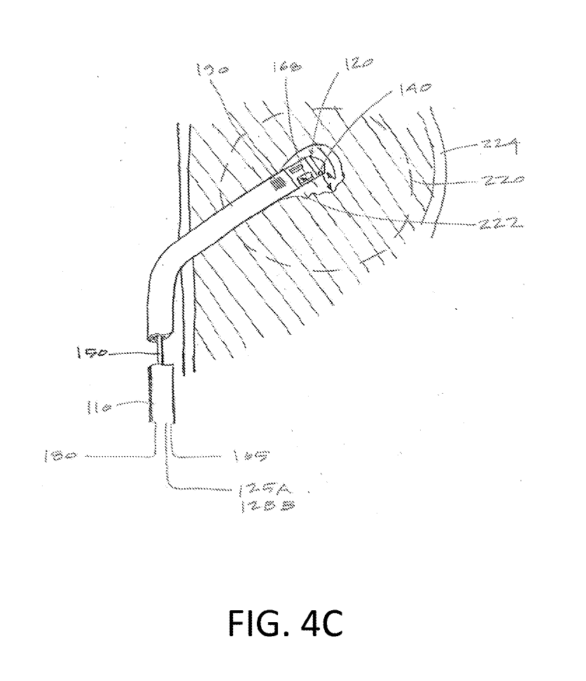

In one variation shown in FIG. 1, the sleeve 110 has a diameter of approximately 3 to 6 mm and is formed of a high strength polymer tubing (e.g., PEEK) that is flexible and has a repose curved shape 128 in the distal region. The probe sleeve 110 has a length that is adapted to flex and straighten to allow it introduction through the working channel of an endoscope or cystoscope 130 used in transurethral access of a male patient's prostate gland. The curved shape 128 of the shaft allows it to be introduced through the patient's urethra 132 and interstitially into prostate tissue 134 (see FIGS. 4A-4B).

In one variation, the probe sleeve 110 is introduced over a guidewire 138 as shown in FIGS. 4A-4B.

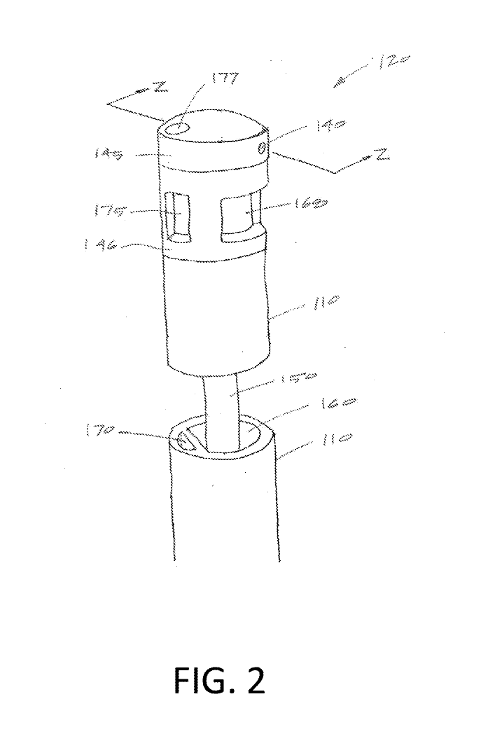

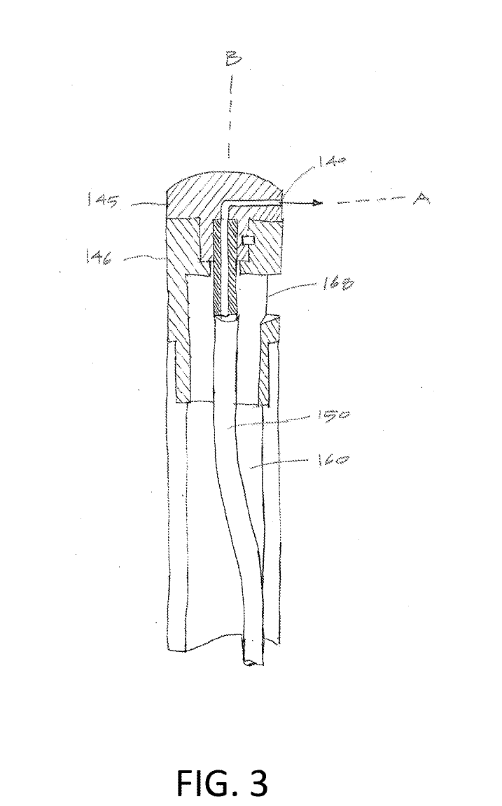

In one aspect, the instrument can be adapted for cutting tissue with a fluid jet, for example to treat Benign Prostatic Hyperplasia (BPH). Now turning to FIGS. 2 and 3, the working end 120 carries a jetting port 140 that communicates with the high pressure media source 125A wherein the axis A of the jetting port is approximately perpendicular to the axis B of the working end 120, or the jetting axis A can be about 45° to 90° from axis B. As can be seen in FIGS. 2-3, the working end 120 has a jetting body 145 that is rotatable relative to proximal body 146 to allow the physician to move the jetting port 140 when in targeted tissue as will be described below. The proximal body 146 is attached to the shaft member or sleeve 110. The jetting body 145 is fixedly coupled to high-strength flexible polymer sleeve 150 that carries the flow media from source 125A to the jetting port. Referring back to FIG. 1, the flexible sleeve 150 is fixed to rotatable handle portion 152A which can be rotated relative to the stable handle portion 152B. The flexibility and torque resistance of the sleeve 150 is adapted to allow for easy rotational movement of jetting body 145 either in 360° or back and forth from a few degrees to 180° or more.

In another aspect, the probe can be adapted to extract fluid media and resected tissue from a treatment site. In FIGS. 1 and 3, it can be seen that sleeve 150 is loosely carried in extraction channel 160 in the probe sleeve 110. Thus, the extracting channel 160 is adapted to extract and remove fluid media and resected and pulverized tissue from a treatment site as will be described below. At the working end 120, the extraction channel communicates with opening 168 in body 146 (FIG. 2). The extracting channel 160 extends through sleeve 110 and handle portion 152E to extraction tubing 162 that is operatively coupled to a negative pressure source 165 (see FIG. 1) and a collection reservoir 166.

In another variation, a probe, as shown in FIGS. 1 and 2, includes a separate pressure sensing channel 170 that extends through the probe sleeve 110 to an open termination 175 in body 146. The channel 170 extends proximally through handle portion 152B to tubing 172 that is in fluid communication with a pressure sensor 180 to thus allow direct pressure sensing in the cavity that is being resected in a targeted tissue site. The channel 170 and tubing 172 may be purged prior to resecting tissue to insure a liquid column extending from open termination 175 to the pressure sensor 180. Any such sensor is operatively coupled to the controller 125B.

Still referring to FIGS. 1-2, the pressure sensing channel 170 can extend through probe sleeve 110 and handle portions 152A and 152B to allow a guidewire 138 to pass through the channel 170 and channel portion 170′ in the handle from proximal opening 176 to distal opening 177 (FIGS. 1-2). In order for the guidewire 138 to be used, the relative rotational positions of working end bodies 145 and 146 must be aligned and the handle portion can have an indent mechanism and indicators 178, 178′ to indicate alignment of the channels in components 145, 146, 150, 152A and 152B to permit passage of the guidewire 138. After use of the guidewire 138, as will be described below, a valve 184 in the handle can be closed, which can be a flap valve, duck-bill valve, manual valve or the like provide a fluid tight flow to the pressure sensor 180.

In another embodiment, a fiber optic pressure sensor or other similar sensor on the distal end of an elongate wire-like element can be introduced through the channel 170 to position the sensor at the treatment site.

In another aspect, the jetted fluid media in can be water entrained with a bioresorbable particulate media including, but not limited to ice, bio-absorbable particles, bio-resorbable particles or bio-degradable particles (e.g. poly(glycolic acid) (PGA), poly(L-lactic acid), poly(ortho ester) (POE), poly(epsilon-caprolactone) (PCL)). The particles entrained in the jetted liquid can function as an abrasive and cause more rapid tissue dissection, resection and pulverization. The particulate media can introduced into a stream of water at or proximate to source 165 and then flow through the supply line 122 into and through the handle 105 to the working end 120 of the instrument (FIG. 1).

In one aspect of the invention relating to the jetted flow media, when the particulate material is ice, the jetted fluid and ice particles can be generated or provided by source 165 in a proximal region of the flow path before the supply line 122 enters the handle 105 of the instrument (FIG. 1).

The jetted flow media can consist of a flow of cryogenic media which is a liquid in source 165 as remains a liquid as the media flows through an insulated, high pressure sleeve 150 and is ejected from port 140. As the cryogenic media exits the port 150 and interacts with tissue, it vaporizes and freezes or cools tissue while at the same time locally cooled tissue more fracturable and pulverizable by the cryogenic media as it interacts with the tissue. This cryogenic flow media also could contain bioresorbable particulate media as described above.

In another aspect of the invention relating to jetted flow media, the flow media can consist of first and second flows of media through a dual lumen sleeve 150 (not shown; cf. FIGS. 1-3) wherein the first flow is a cryogenic media from source 165 as described above and the second flow is water to provide a water jet for cutting tissue as described above. The first and second flows can exist a single port 140 in the working end or adjacent ports. The dual lumens in sleeve 150 can be side-by-side or concentric and the exit ports likewise case proximate to one another or concentric. In this embodiment, the controller can independently control the flows, flow sequences, pulse rates, velocities and other flow parameters. As one example, the cryogenic flows can precede pulses or intervals of water jets to freeze larger portions of the tissue. Thus, a method of the invention includes a first step of introducing a cryogenic media to interact with targeted tissue to cause change in tissue characteristics (e.g., freezing the tissue) to make the treated tissue more susceptible to high pressure fluid jet cutting. A subsequent step includes jetting a high-pressure fluid column into the treated tissue to fracture, dissect and pulverize the tissue, wherein the treated tissue is thus more treatment-susceptible due to the cryogenic pre-treatment. This type of cryogenic pre-treatment is useful is tissues that may be resistant to water jet cutting.

In another variation, a method of the invention includes a first step of pre-treating the targeted tissue with RF, resistive or other thermal treatment means to cause a change in tissue characteristics again to make the targeted tissue more susceptible to high pressure fluid jet cutting. In this aspect of the invention, the working end 120 can carry Ili-polar electrodes or another form of heat emitter to treat the tissue. The thermal energy means can include RF electrodes, resistive heaters, microwave means, laser, inductive or ultrasound. Again, the subsequent step includes jetting a high-pressure fluid column into the heated and altered tissue to fracture, dissect and pulverize the tissue, wherein the altered tissue is thus more treatment-susceptible due to the thermal pre-treatment.

In another embodiment, the working end can contain a vibrator element 190 (see FIG. 5) which can vibrate the working end to quickly move the fluid jet about a treatment site to insure that the jet is not focused on one location for more than an instant. The vibratory element 190 can be of the type found in cell phones and can be coupled to a low voltage source and controlled by controller 125B.

Now turning to FIGS. 4A-4B, the instrument 100 is shown in a urology procedure adapted to resect and extract prostate tissue to treat BPH. In FIG. 4A, an endoscope 30 is introduced through the urethra 200 into a patient prostate 204. The distal end of the endoscope is navigated to an in the prostate by using landmarks as known in the art. Thereafter, the sleeve 110 of the instrument 100 is introduced through a working channel in the endoscope 30 until the working end is exposed beyond the endoscope 30. Then, a guidewire 138 is introduced through the probe 100 and a curved tip of the guidewire 138 is penetrated through the urethra 200 to a targeted treatment site 210 a lobe of the prostate 204 under endoscopic viewing. The distal positioning of the guidewire tip can be viewed with a TRUS system (trans-rectal ultrasound) as is known in the art.

FIG. 4B illustrates a subsequent step of invention wherein the probe working end 120 is advanced over the guidewire 138 to the targeted site 210. Next, referring to FIG. 4C, the physician actuates the probe/controller which caused fluid a flow media as described above to jet under high pressure from port 140 to thereby resect, pulverize and obliterate tissue proximate to the port 140. Contemporaneous with actuation of the jetting the flow media, the controller actuates the negative pressure source 165 to suction tissue against the suction port 168 which bring the tissue into contact with the jetted fluid and at the same time suction the flow media and tissue debris through the extraction channel 160 of the probe sleeve 110 to the collection reservoir 166. At the same time, the physician can manually rotate the working end body 145 to resect tissue and also manually translate the working end 120 axially to resect the targeted tissue. In FIG. 4C, it can be seen that a region 220 can be resected and extracted by this method. All of the steps of resecting tissue can be observed intra-operatively by a TRUS system. The system and method provides a probe that can operate to cut and extract tissue at a rate of at least 5 grams per minute. The working end can be moved to a plurality of locations in both lobes of the prostate to remove tissue by repeating the steps above.

In another variation, the vibratory mechanism 190 can be actuated contemporaneous with the fluid jetting. In any of the methods, the working end and jet thus can be configured to rotate, translate axially, reciprocate, oscillate and/or vibrate. In another embodiment, the system can be motorized to rotate body 145 and jetting port 140 at a rate of between 10 rpm and 1,000 rpm.

Still referring to FIG. 5, while operating the probe to resect and extract tissue, the pressure sensor 180 coupled to controller 125B can monitor the interstitial pressure about the cavity 222 being. created and a particular change in such interstitial cavity pressure can indicate a leak or perforation of jet through the capsule 224 of the prostate 204. Further, in response to sensing fluid pressure at the targeted site, the controller can have algorithms to modulate or terminate an operating parameter of the media flow in response to sensed fluid pressure in the site.

It should be noted that all the aforementioned components may be essential to the invention alone or in any combination. It should be further be noted that the jetting probe 100 according to the invention can be provided as a separate component and/or can be used with a RF device, resistive heating device or the like for tissue coagulation.

In general, cutting and removing a target tissue can include the steps of introducing a working end of a probe into an interstitial site in a patient body, activating a pressure source to provide a flow of a cryogenic fluid media through the probe and jetting the fluid media from the working end at operating fluids configured to cut tissue and activating a negative pressure source coupled to a flow channel in the probe to thereby extract cut tissue and fluid media from the site. The method further comprises sensing fluid pressure at the site and modulating an operating parameter of the flow in response to sensed fluid pressure in the site. The method can de-activate the pressure source if the sensed pressure drops below a predetermined level or the method can include de-activating the pressure source if the sensed pressure drops below a predetermined level for a selected time interval.

In another aspect, a method comprises cutting and removing a target tissue with the steps of introducing a working end of a probe into an interstitial site in a patient body, activating a pressure source to provide a flow of a fluid media through the probe and jetting the fluid media from the working end at operating parameters configured to cut tissue, activating another pressure source to provide a flow of a cryogenic media through the probe and jetting the cryogenic media from the working end to interact with tissue and activating a negative pressure source coupled to a flow channel in the probe to thereby extract cut tissue and media from the site. The method further comprises sensing fluid pressure at the site and modulating an operating parameter of the flow in response to sensed fluid pressure in the site.

In another aspect, a method comprises cutting and removing a target tissue with the steps of introducing a working end of a probe into an interstitial site in a patient body, activating a pressure source to provide a flow of a fluid media through the probe and jetting the fluid media from the working end at operating parameters configured to cut tissue and sensing fluid pressure at the interstitial site and modulating an operating parameter of a flow of fluid media in response to sensed fluid pressure in the interstitial site.

In another variation, the working end 120 can be provided with a plurality of jetting orifices. Another variation can have an articulating probe working end actuated by a pull wire or the like as is known in the art.

In another variation, the working end can carry one or more hyper-echoic elements or features for cooperating with an ultrasound imaging system to permit better imaging intra-operatively.

In another variation, the system can include an adhesive source configured for introducing a tissue adhesive through the probe working end to the targeted site 222.

Thereafter, the negative pressure source 165 can be used to collapse the interstitial cavity and adhere the cavity walls together. In one variation, the tissue adhesive is at least in part a cyanoacrylate.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A method of cutting and removing a target tissue, comprising:
introducing a working end of a probe into an interstitial site in a patient body;
activating a pressure source to provide a flow of a cryogenic media through the probe and jetting the cryogenic media from the working end at an operating parameter configured to mechanically remove tissue;
contemporaneously moving the working end; and
activating a negative pressure source coupled to a flow channel in the probe to extract the removed tissue and cryogenic media from the site.

2. The method of claim 1 wherein moving the working end includes at least one of rotating, translating axially, reciprocating, oscillating and vibrating.

3. The method of claim 2 wherein moving the working end in provided by a motor drive.

4. The method of claim 1 further comprising sensing fluid pressure at the site and modulating an operating parameter of the flow in response to sensed fluid pressure in the site.

5. The method of claim 1 wherein the cryogenic media includes a particulate material selected from the group of ice, biocompatible materials and bioresorbable materials.

6. The method of claim 1, further comprising pre-treating the interstitial site to cause a thermal change in tissue prior to activating the pressure source.

7. The method of claims 4 further comprising monitoring a fluid pressure at the interstitial site using a pressure sensor in communication with a static fluid column extending to the interstitial site.

8. The method of claim 7 wherein the static fluid column extends through an independent channel in the probe.

9. The method of claims 4 further including de-activating the pressure source if the sensed pressure drops below a predetermined level.

10. The method of claims 4 further including de-activating the pressure source if the sensed pressure drops below a predetermined level for a selected time interval.

11. The method of claims 4 wherein the fluid pressure is monitored by a pressure sensor positioned proximate to the site.

12. The method of claim 11 wherein the pressure sensor is carried by the probe.

13. The method of claim 11 wherein the pressure sensor is carried by a member insertable through a channel in the probe.

14. The method of claim 8 wherein the independent channel has a diameter of at least 0.5 mm.

15. The method of claim 1 wherein the probe is operated to cut tissue at a rate of at least 5 grams per minute.

16. The method of claims 1 wherein the probe extracts cut tissue through an extraction channel in the probe.

17. The method of claim 2 wherein the working end is rotated at a rate of between 10 rpm and 1,000 rpm.

18. The method of claims 1 wherein the cryogenic media is jetted from the working end through at least one jetting orifice.

19. The method of claims 1 wherein the introducing step includes advancing the working end over a guide member to the site.
